(19) United States
(12) Patent Application Publication
(10) Pub. No.: US 2026/0328680 A1
(43) Pub. Date: Oct. 22, 2026

(54) Investigating a Textile Fiber Structure Containing Two Components

(71) Applicant: Uster Technologies AG, Uster (CH)

(72) Inventor: Rainer Jacob, Fehraltorf (CH)

(73) Assignee: Uster Technologies AG, Uster (CH)

(21) Appl. No.: 18/993,384

(22) PCT Filed: Aug. 8, 2022
(86) PCT No.: PCT/CH2022/000005

§ 371 Date: Jan. 10, 2025

(51) Int. Cl. G01N 21/3563; G01N 33/36; G06T 7/00; G06V 10/60

(52) U.S. Cl. CPC G01N 21/3563; G01N 33/36; G06T 7/0004; G06T 2207/10048; G06T 2207/30124; G06V 10/60

(57) ABSTRACT

System consisting of a textile fiber structure containing two components and a device for investigating the textile fiber structure. The device contains a radiation source for transmitting electromagnetic radiation in a spectral band in the direction of the textile fiber structure, a radiation sensor for receiving the electromagnetic radiation, and a spectral filter having spectral properties in the spectral band for filtering the electromagnetic radiation. The transmittance of the spectral filter, in the spectral band has at least one local maximum and at least one local minimum. The spectral properties of the spectral filter in the spectral band are matched to the spectral properties of the radiation source and each of the two components such that a radiation intensity received by the radiation sensor is a monotonic function of the mixing ratio of the two components. The device is simply constructed and allows for the use of spatially resolving image sensors.

### FIELD OF THE INVENTION

The present invention is in the field of quality monitoring in the textile industry. It relates to a system consisting of a textile fiber structure containing two components and a device for investigating the textile fiber structure, according to the first claim. It also relates to a device and a method for examining a textile fiber structure containing two components, according to the further independent claims. Preferred applications are the detection of foreign materials in a textile fiber structure such as fiber flocks, fiber web, sliver, roving, yarn, woven or knitted fabric and the determination of a mixing ratio of two components of such textile fiber structures.

### DESCRIPTION OF THE PRIOR ART

Foreign materials in the yarn represent one of the major problems of today's spinning mills. These are materials that differ from the base material of the yarn fibers, e.g., cotton fibers. They can be of various origins, such as residues of transport packaging (plastic packaging, cords), civilization impurities (soot parts, plastic bags) or residues of living beings (human or animal hair, plant stalks). Foreign materials cause thread breakage during spinning and weaving, take dye in a different way than the base material and affect the appearance of the final textile product. They significantly reduce the value of the final product. An overview of fabric defects caused by foreign materials and recommendations to reduce them is given in paragraph 3.8 of the USTER® NEWS BULLETIN NO. 47 “The origins of fabric defects—and ways to reduce them”, Uster Technologies AG, March 2010.

Foreign materials can be detected and, if necessary, rejected at various stages of the yarn manufacturing process.

The blowroom process is part of the yarn manufacturing process and is upstream of the carding process. The aim is to prepare the raw material so that it can be fed to the carding process in as constant a quality as possible and free of impurities. It comprises the opening of the raw material, the feeding of the same into the processing operation, and the mixing and coarse cleaning of the fed material. Depending on the design of the process, individual work steps can be run through several times or even omitted. At this stage of the process, the material is in the form of fiber flocks (for example, in the case of cotton and wool) or shreds (in the case of synthetic fiber material). The material is transported by an air stream that connects the various units in the blowroom process.

The spinning process is another part of the yarn manufacturing process and is indirectly or directly downstream of the carding process. In this process, the yarn as the end product is spun from a sliver, e.g., the intermediate product of a carding machine, or a roving. In the process, the roving or sliver is transformed into its final form, the yarn, by drawing and twisting. During spinning, the yarn is wound onto spindles. Subsequently, the spindles are rewound onto large bobbins. The material is transported in the form of spindles and bobbins.

The purification of foreign materials can basically be divided into the following three steps:
1. Detection of the foreign material;
2. Spatial/temporal localization of the foreign material within the test material; and
3. Ejection of the foreign material.

In the blowroom process, foreign material cleaning can be carried out manually before the raw material is fed to the automatic converting process, or cleaning can be carried out automatically by appropriate equipment within the blowroom process. Nowadays, automatic cleaning is common.

In the case of automatic cleaning, detection and localization are carried out with the aid of detection devices which recognize differences in a certain characteristic within the material flow. The following are mentioned in a non-exhaustive way here: reflection and transmission of electromagnetic radiation or fluorescence. In the simplest applications, optical detection devices are used to mimic the human eye and analyze the color impression of the material stream, detecting corresponding color differences. U.S. Pat. No. 6,452,157 B1 discloses a device for detecting impurities, foreign materials and foreign fibers in textile fiber material. The device has at least two light sources which alternately illuminate the fiber material with different colors. Furthermore, a sensor is provided which receives the colors of the light reflected from the fiber material.

However, for the detection of foreign materials that are transparent to visible light or have a similar color to the raw material, more sophisticated detection devices are needed. In this case, the material flow can be analyzed with the help of such electromagnetic radiation which is not perceived by the human eye (ultraviolet or infrared). In this case, the material affiliation is determined on the basis of characteristic signatures (for example, sequence of specific absorption bands) in the reflected or transmitted spectrum of the electromagnetic radiation. The more characteristics (e.g., absorption bands) within the signature are used for discrimination, the more accurate the discrimination of the characteristic signatures becomes. Currently, each characteristic within the signature requires a dedicated sensor within the detection device, so it only responds to the presence/absence of that one characteristic. The more characteristics that are to be used, the more complex the detection device becomes. The input signal must be split accordingly to the number of sensors and thus loses intensity. Furthermore, for certain characteristics there are currently no spatially, but only temporally resolving sensors. This requires a device upstream of the detection device that links time and location. Alternatively, the incident electromagnetic radiation can be modulated in time to match the characteristics. However, for the non-visible range of the electromagnetic spectrum, this is sometimes associated with great effort.

EP-1'961'848 A1 discloses a device for detecting and separating foreign materials from a material stream of raw cotton. The device has an irradiation device for irradiating the material stream and a sensor device for detecting irradiated foreign materials in the material stream. The irradiation device can emit visible light, ultraviolet or infrared radiation. A rotatable polygon mirror is provided for scanning the material stream line by line, with which the beam of the irradiation device can be deflected in the direction of the material stream and with which the beam reflected by the material stream can be deflected towards the sensor device. The irradiation device and the sensor device are arranged coaxially one behind the other in relation to an optical radiation axis. The irradiation device essentially consists of a radiation source, a concave mirror for aligning and focusing the radiation along the radiation axis to the polygon mirror, and a rear reflector, with which a rear part of the radiation can be guided in the direction of the concave mirror.

When distinguishing the material on the basis of color differences, as is common in the spinning process, the characteristics mentioned lie in the visible spectral range. Since the color impression is also due to the specific reflection/transmission of certain parts of the irradiated wavelength spectrum, each characteristic must also be detected individually in this case. For this purpose, either a temporal color modulation of the input signal can be performed, or the output signal modified by the yarn must be decomposed into the individual characteristics, as already explained. In the former case the spatial resolution deteriorates, in the latter the signal-to-noise ratio. The more colors are used, the greater the effect of these disadvantages. For this reason, processes have become established in practice that use only one or at most two colors. A yarn clearer that scans the yarn with several differently colored light components is known from WO-2011/026249 A1.

Multivariate optical filters are a special category of optical transmission filters or reflection filters. The filter characteristics of multivariate optical filters are adapted to a specific chemical signature. Different characteristics of the signature can be used simultaneously and independently in a single filter. Multivariate optical elements thus allow material identification based on the chemical signature. If the input signal to the filter exactly matches the matched signature, the signal passes through the multivariate optical filter unimpeded. If the input signal deviates from the signature, it is attenuated as it passes through the filter. The greater the deviation, the greater the attenuation. Multivariate optical filters, in addition to discriminating materials, also allow a mixing ratio to be determined based on the chemical signature that is altered by the mixing. The specific transmittance or reflectance of multivariate optical filters is obtained from the chemical signatures of the materials to be discriminated using the partial least squares method. An example of multivariate optical filters can be found in R. J. Priore and N. Jacksen, “Spectral imaging of chemical compounds using multivariate optically enhanced filters integrated with InGaAs VGA cameras”, Proc. of SPIE Vol. 9824, S. 98240P-1 bis 98240P-10, 2016.

US-2017/0241839 A1 proposes using several integrated computing elements in combination with each other to improve the detection sensitivity of optical computing devices. The optical computing device disclosed therein includes two or more integrated computing elements formed as multivariate optical filters. The integrated computing elements are identical to each other and interact optically one after the other with incident electromagnetic radiation. Further, the optical computing device includes a detector which receives those photons which have interacted optically with each integrated computing element.

### SUMMARY OF THE INVENTION

It is the object of the present invention to create a system and a device for investigating a textile fiber structure containing two components, which avoids the above disadvantages. In particular, the system and the device are to be of simple construction. They should enable a high spatial resolution and the use of imaging, spatially resolving radiation sensors. At the same time, the signal-to-noise ratio shall be high. A further object is to provide a corresponding method for investigating a textile fiber structure containing two components.

A further object is to provide a system, a device and a method for detecting foreign materials in a textile base material which avoid the above disadvantages. A still further object is to provide a system, a device and a method for determining a mixing ratio of two components of a textile fiber structure, which avoid the above disadvantages.

These and other objects are solved by the system according to the invention, the device according to the invention and the method according to the invention, as defined in the independent claims. Advantageous embodiments are indicated in the dependent claims.

The invention is based on the idea of designing a spectral filter specifically with respect to the two components of the textile fiber structure, so that a radiation intensity received by the radiation sensor is a monotonic and preferably linear function of the mixing ratio of the two components. Furthermore, the transmittance or reflectance in the spectral band under consideration is to have at least one local maximum and at least one local minimum such that multiple characteristic wavelengths are considered. Furthermore, the invention comprises that said spectral filter can also be designed for the ratio of different characteristics, e.g. color impressions, and thus also makes color differences of materials of the same type detectable without signal loss.

The textile fiber structure may contain two or more components, wherein the invention is directed to two specific components in each case. The two components differ from each other in terms of their chemical or optical properties.

According to the invention, the design of the spectral filter is based on specific chemical and/or color signatures of the two components. Characteristics of one component can be amplified by high transmission and produce a high output signal, while characteristics of the other component can be attenuated by low transmission and produce a low output signal. The combination of amplification and attenuation in the spectral filter makes it possible to differentiate between the two components on the basis of the signal emerging from the spectral filter and thus to investigate the textile fiber structure with regard to the two components.

If the textile fiber structure is investigated as a unit, without taking local inhomogeneities into account, the invention provides as a result the proportional contribution of the two components, which can be interpreted as a mixing ratio. Thus, the invention allows the determination of a mixing ratio of the two components of the textile fiber structure.

If, on the other hand, a spatially resolving image sensor is used, the local differences between the two components are clearly visible in the image it produces. If the proportion of the first component in the textile fiber structure predominates and the other, second component occurs only sporadically and is undesirable, the first component is referred to as the base material and the second as the foreign material. According to the invention, the spectral filter is designed in such a way that it amplifies characteristics of one component (e.g., the foreign material) and attenuates characteristics of the other component (e.g., the base material). As a result, foreign material and base material are imaged with different intensities, which enables good differentiation of the components and thus detection of the foreign material in the base material.

The system according to the invention consists of a textile fiber structure containing two components and a device for examining the textile fiber structure. The device includes a radiation source for emitting electromagnetic radiation in a spectral band in the direction of the textile fiber structure to interact with the textile fiber structure. Further, the device includes a radiation sensor for receiving at least a part of the electromagnetic radiation after interaction with the textile fiber structure. The device also includes a spectral filter having spectral properties in the spectral band for filtering at least a part of the electromagnetic radiation before or after interaction with the textile fiber structure. The transmittance or reflectance of the spectral filter in the spectral band has at least one local maximum and at least one local minimum. The spectral properties of the spectral filter in the spectral band are adapted to the spectral properties of the radiation source and each of the two components such that a radiation intensity received by the radiation sensor is a monotonic function of the mixing ratio of the two components.

The two components of the textile fiber structure are, for example, two different elements from the following set: cotton, linen, virgin wool, polyethylene (PE), polypropylene (PP), polyvinyl chloride (PVC), polyester (PES); polyacrylic (PAN); viscose (CV, regenerated cellulose), modal (CMD), lyocell (CLY), polyethylene terephthalate (PET), polystyrene (PS), polyamide (PA), acrylonitrile-butadiene-styrene copolymer (ABS), polymethyl methacrylate (PMMA), polyoxymethylene (POM), elastane (EL), aramid (AR), acetate (CA), cupro (CUP). Preferably, the two components of the textile fiber structure are a pair from the following set: cotton and polyethylene (PE), cotton and polypropylene (PP), cotton and polyvinyl chloride (PVC), cotton and polyester (PES); cotton and polyacrylic (PAN); cotton and viscose (CV, regenerated cellulose), cotton and polyethylene terephthalate (PET), cotton and polystyrene (PS), cotton and polyamide (PA), cotton and acrylonitrile-butadiene-styrene copolymer (ABS), cotton and polymethyl methacrylate (PMMA), cotton and polyoxymethylene (POM).

The device according to the invention is used to examine a textile fiber structure containing two components. The device contains a radiation source for transmitting electromagnetic radiation in a spectral band in the direction of the textile fiber structure for interaction with the textile fiber structure. Further, the device contains a radiation sensor for receiving at least a portion of the electromagnetic radiation after interaction with the textile fiber structure. The device also contains a spectral filter having spectral characteristics in the spectral band for filtering at least a portion of the electromagnetic radiation before or after the interaction with the textile fiber structure. The two components of the textile fiber structure are two different elements from the following set: cotton, linen, virgin wool, polyethylene (PE), polypropylene (PP), polyvinyl chloride (PVC), polyester (PES); polyacrylic (PAN); viscose (CV, regenerated cellulose), modal (CMD), lyocell (CLY), polyethylene terephthalate (PET), polystyrene (PS), polyamide (PA), acrylonitrile-butadiene-styrene copolymer (ABS), polymethyl methacrylate (PMMA), polyoxymethylene (POM), elastane (EL), aramid (AR), acetate (CA), cupro (CUP). The transmittance or reflectance of the spectral filter in the spectral band has at least one local maximum and at least one local minimum. The spectral properties of the spectral filter in the spectral band are matched to the spectral properties of the radiation source and each of the two components such that a radiation intensity received by the radiation sensor is a monotonic function of the mixing ratio of the two components.

In one embodiment of the device, the two components of the textile fiber structure are a pair from the following set: cotton and polyethylene (PE), cotton and polypropylene (PP), cotton and polyvinyl chloride (PVC), cotton and polyester (PES); cotton and polyacrylic (PAN); cotton and viscose (CV, regenerated cellulose), cotton and polyethylene terephthalate (PET), cotton and polystyrene (PS), cotton and polyamide (PA), cotton and acrylonitrile-butadiene-styrene copolymer (ABS), cotton and polymethyl methacrylate (PMMA), cotton and polyoxymethylene (POM).

In one embodiment of the system or device, the at least one local maximum lies at the wavelength or wavelengths of the electromagnetic radiation at which the absolute value of the difference in the absorptance, transmittance, or reflectance of the two components has a local maximum.

In one embodiment of the system or device, the transmittance or reflectance of the spectral filter in the spectral band has at least two local maxima and local minima each.

The spectral filter in the system or device can be designed as a reflection filter or as a transmission filter.

In one embodiment of the system or device, the spectral filter is designed as an interference filter.

In one embodiment of the system or device, the spectral filter is integrated into the radiation sensor.

In one embodiment of the system or device, the spectral characteristics of the spectral filter in the spectral band are matched to the spectral characteristics of the radiation source and each of the two components such that a radiation intensity received by the radiation sensor is a linear function of the mixing ratio of the two components.

One embodiment of the system or device contains an optical imaging system for imaging the textile fiber image onto the radiation sensor, wherein the radiation sensor is spatially resolving and designed either as a digital camera with a two-dimensional image converter or as a one-dimensional line sensor.

One embodiment of the system or device contains a time-varying optical imaging system which images various locations of the textile fiber structure onto the radiation sensor one after the other over time, wherein the radiation sensor is time-resolving (see EP-1'961'848 A1).

The system or device according to the invention can be used to detect a foreign material in a base material, wherein the foreign material and the base material are the two components of the textile fiber structure.

The system or device according to the invention can be used to determine a mixing ratio of the two components of the textile fiber structure.

The method according to the invention is used to investigate a textile fiber structure containing two components. Electromagnetic radiation in a spectral band is transmitted from a radiation source in the direction of the textile fiber structure. At least a part of the electromagnetic radiation interacts with the textile fiber structure. At least a part of the electromagnetic radiation is received by a radiation sensor after interacting with the textile fiber structure. At least a part of the electromagnetic radiation is filtered by a spectral filter having spectral properties in the spectral band before or after interacting with the textile fiber structure. The spectral filter is selected such that its transmittance or reflectance in the spectral band has at least one local maximum and at least one local minimum, and its spectral properties in the spectral band are adapted to the spectral properties of the radiation source and each of the two components in the textile fiber structure such that a radiation intensity received by the radiation sensor is a monotonic function of the mixing ratio of the two components.

In one embodiment of the method, the two components of the textile fiber structure are two different elements from the following set: cotton, linen, virgin wool, polyethylene (PE), polypropylene (PP), polyvinyl chloride (PVC), polyester (PES); polyacrylic (PAN); viscose (CV, regenerated cellulose), modal (CMD), lyocell (CLY), polyethylene terephthalate (PET), polystyrene (PS), polyamide (PA), acrylonitrile-butadiene-styrene copolymer (ABS), polymethyl methacrylate (PMMA), polyoxymethylene (POM), elastane (EL), aramid (AR), acetate (CA), cupro (CUP). Preferably, the two components of the textile fiber structure are a pair from the following set: cotton and polyethylene (PE), cotton and polypropylene (PP), cotton and polyvinyl choride (PVC), cotton and polyester (PES); cotton and polyacrylic (PAN); cotton and viscose (CV, regenerated cellulose), cotton and polyethylene terephthalate (PET), cotton and polystyrene (PS), cotton and polyamide (PA), cotton and acrylonitrile-butadiene-styrene copolymer (ABS), cotton and polymethyl methacrylate (PMMA), cotton and polyoxymethylene (POM).

The method according to the invention can be used to detect a foreign material in a base material, wherein the foreign material and the base material are the two components of the textile fiber structure.

The method according to the invention can be used to determine a mixing ratio of the two components of the textile fiber structure.

In the system, device or method, the spectral band can be in the wavelength range between 300 nm and 2200 nm, for example. However, the spectral band can be chosen arbitrarily if appropriate manufacturing processes are available for the multi-variate filter. The wavelength range between 700 nm and 1900 nm has proven to be advantageous for the investigation of material pairings with the base material cotton and foreign materials such as polyester, polyacrylic, polypropylene and polyethylene.

In the system, device or method, the spectral band can have a width between 100 nm and 1500 nm, for example, and preferably between 300 nm and 500 nm.

The textile fiber structure can be present, for example, in the form of fiber flocks, fiber web, sliver, roving, yarn, woven or knitted fabric.

The device according to the invention can, for example, be used inline in a textile production plant, e.g., as a fiber cleaner or as a yarn clearer, or offline in a textile laboratory, e.g., as a fiber testing device or as a yarn testing device. The same applies to the method according to the invention.

The radiation source may contain a single radiation element, e.g., a halogen lamp. Alternatively, it can contain several radiation elements, e.g. a halogen lamp and a mercury vapor lamp.

According to the invention, the spectral filter can be used in transmission or reflection. A single or several spectral filters can be used in combination with each other (cf. US-2017/0241839 A1).

The expressions “local maximum”, “local minimum” and “monotone function” used in this specification are used in the sense of their respective mathematical meanings. They are known to the person skilled in the art and their definitions can be taken from textbooks or reference works on mathematics.

The system according to the invention, the device according to the invention and the method according to the invention allow a simple and yet reliable determination of a mixing ratio of two components of a textile fiber structure. They avoid splitting the incoming electromagnetic radiation reflected or transmitted by the textile fiber structure into several radiation sensors. The system and the device are simple in design and allow the use of imaging, spatially resolving radiation sensors. They do not require temporal modulation of the input signal, thus achieving a high spatial resolution.

Surprisingly, it has also been shown that the same device according to the invention can be used for various foreign materials. Many of the foreign materials such as polystyrene, polyethylene or polypropylene found in natural textile base materials such as cotton can be detected with one and the same device according to the invention, provided that a suitable spectral band is selected. It is therefore not necessary to design and build a dedicated spectral filter for each foreign material; a single radiation source, a single radiation sensor and a single spectral band are also sufficient.

### BRIEF DESCRIPTION OF THE DRAWINGS

In the following, an embodiment of the invention is explained in detail with reference to the drawings. For the sake of a clearer illustration, an application is described in which a proportion of a foreign material in a base material of a textile fiber structure is determined. However, this is not intended to limit the generality of the invention. The invention can alternatively be directed to a textile fiber structure with two desired components. The invention may relate to the detection of a foreign material in a base material or to the determination of a mixing ratio of two components of the textile fiber structure.

FIG. 1 schematically shows an embodiment of the system and device according to the invention.

FIG. 2 shows various spectra in a common first spectral band, namely: (a) relative intensity distribution of a halogen lamp; (b) absorptance of cotton; (c) absorptance of polyethylene; and (d) transmittance of a spectral filter.

FIG. 3 shows a relative intensity distribution of a halogen lamp in a second spectral band.

FIGS. 4-10 show absorption coefficients of the materials cotton, polyethylene, polypropylene, polyester, polyethylene terephthalate, polyacrylic and cellulose in the second spectral band.

FIG. 11 shows a transmittance of a spectral filter in the second spectral band.

FIG. 12 shows (a) a grayscale image of a textile sample with different materials and (b) a binary image of the sample after carrying out the method according to the invention and image processing.

### DETAILED DESCRIPTION OF THE INVENTION

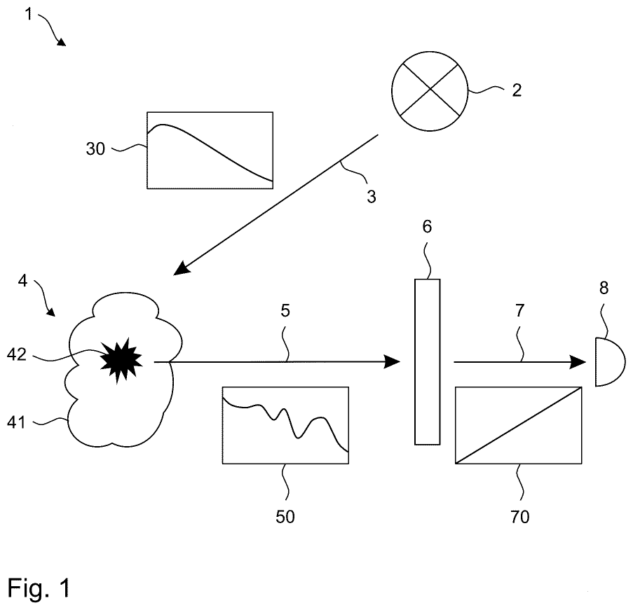

An embodiment of the system according to the invention and the device 1 according to the invention is shown schematically in FIG. 1. The device 1 contains a broadband radiation source 2 for generating electromagnetic radiation 3 in a spectral band. The generated electromagnetic radiation 3 has a spectral intensity distribution 30 in the spectral band which is characteristic of the radiation source 2. In FIG. 1, the intensity distribution 30 is shown as a schematic diagram in which the intensity is plotted as a function of the wavelength.

At least a part of the electromagnetic radiation 3 generated by the radiation source 2 impinges on a textile fiber structure 4 to be examined. The textile fiber structure 4 can be, for example, one or more fiber flocks, a fiber web, a sliver, a roving, a yarn, a woven fabric, a knitted fabric, or a nonwoven. In the example of FIG. 1, without limiting the generality, a fiber flock is schematically drawn as textile fiber structure 4.

The textile fiber structure 4 contains at least two different components 41, 42. Without limiting generality, it is assumed here for illustrative purposes that the textile fiber structure 4 consists of a base material 41, e.g., cotton, and may possibly contain one or more foreign materials 42 that differ from the base material 41. For example, the foreign material 42 can be polyethylene. When the electromagnetic radiation 3 impinges on the textile fiber structure 4, there is an interaction of the electromagnetic radiation 3 with the base material 41 and, if present, the foreign material 42. As a result of the interaction, the spectral intensity distribution 30 of the electromagnetic radiation 3 is changed according to the chemical or color characteristics of the materials. Radiation 5 reflected or transmitted at the textile fiber structure 4 thus has a spectral intensity distribution 50 that differs from the intensity distribution 30 of the radiation 3 impinging on the textile fiber structure 4. The intensity distribution 50 of the reflected or transmitted radiation is again shown in FIG. 1 as a schematic diagram in which the intensity is plotted as a function of wavelength.

After interaction with the textile fiber structure 4, the electromagnetic radiation 5 interacts with a spectral filter 6 in the exemplary embodiment of FIG. 1. The interaction can occur by means of transmission or reflection at the spectral filter 6. The spectral properties of the spectral filter 6 are specifically matched to the base material 41, a type or class of foreign material 42 and the respective spectral band. The spectral filter 6 may be designed as an interference filter, for example. It changes the intensity distribution 50 of the electromagnetic radiation 5 interacting with it in such a way that differences between the base material 41 and the foreign material 42 are amplified.

If the spectral intensity distribution 50 before the spectral filter 6 corresponds to that of the base material 41, then the intensity of a radiation 7 after the spectral filter 6 should be minimal, for example. If, on the other hand, the spectral intensity distribution 50 upstream of the spectral filter 6 corresponds to that of the foreign material 42, the intensity of radiation 7 downstream of the spectral filter 6 shall be, for example, a maximum. If the spectral intensity distribution 50 before the spectral filter 6 has characteristics of both materials 41 and 42, the intensity of the radiation 7 after the spectral filter 6 shall correspond to a monotonic and preferably linear function of the mixing ratio of the materials 41 and 42. This is schematically illustrated in FIG. 1 by a diagram 70 showing an intensity of radiation 7 after interaction with spectral filter 6 as a linear function of the proportion of foreign materials 42 in textile fiber structure 4.

The spectral filter 6 thus converts the incident wavelength-dependent intensity distribution 50 into an intensity distribution 70 that is a monotonic and preferably linear function of the mixing ratio of the two components 41 and 42. The intensity of the electromagnetic radiation 7 present after the spectral filter 6 is thus a measure of the mixing ratio. In the example discussed here, it is a measure of the presence and amount of the foreign material 42 in the textile fiber structure 4 and/or of the degree of color variation between the base material 41 and the foreign material 42.

After interaction with the spectral filter 6, electromagnetic radiation 7 is detected by a broadband radiation sensor 8, which is in any case capable of detecting radiation 7 in the relevant spectral band. The radiation sensor 8 is preferably spatially resolving and time-resolving. It can, for example, be designed as a digital camera or a digital line sensor.

If the radiation sensor 8 is not spatially resolving, the radiation intensity received by it is a measure of the mixing ratio of the two components 41, 42 of the textile fiber structure 4, according to the diagram 70.

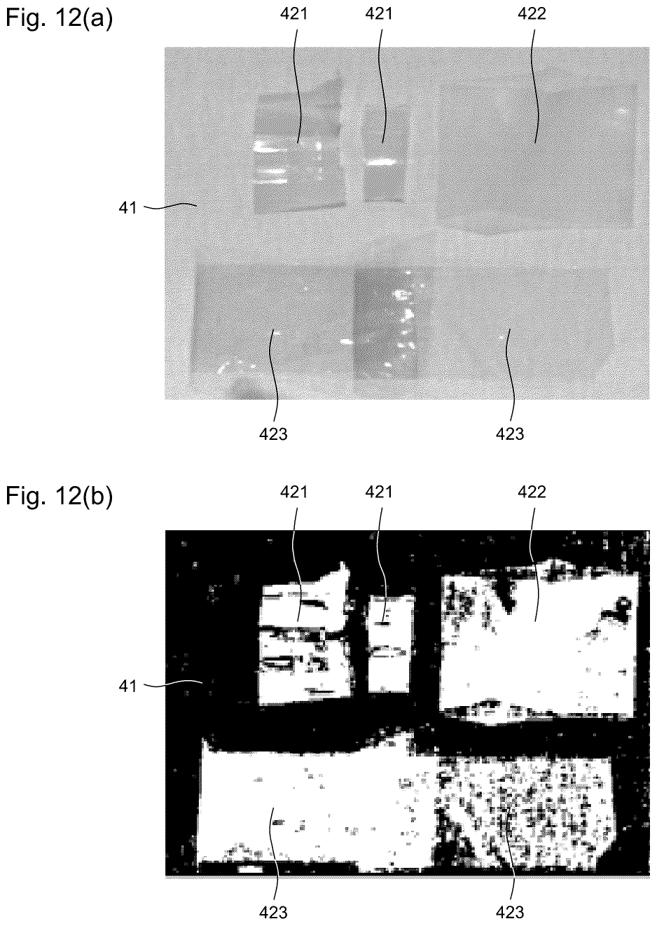

In a preferred embodiment, the radiation sensor 8 is spatially resolving, and the textile fiber structure 4 is imaged onto the radiation sensor 8 by means of optics (not drawn in). This also provides information about the number, position, size and shape of the foreign materials 42 present in the textile fiber structure 4. Thus, the foreign materials 42 can be detected and localized in the textile fiber structure 4. In an image of the textile fiber structure 4 taken by the radiation sensor 8, foreign materials 42 appear bright against a dark background in the present example; see FIG. 12(b).

In an alternative embodiment, the spectral properties of the spectral filter 6 can be matched to the radiation source 2, the base material 41 and/or the foreign material 42 in such a way that the intensity of the radiation 7 after the spectral filter 6 is at a maximum when the textile fiber structure 4 consists only of the base material 41, and decreases as the proportion of foreign material 42 increases. In this case, foreign materials 42 appear dark against a light background.

In another embodiment, the spectral filter 6 can be inserted in the beam path between the radiation source 2 and the textile fiber structure 4. In this case, the electromagnetic radiation 3 generated by the light source 2 first interacts with the spectral filter 6 and then impinges on the textile fiber structure 4. The effect is analogous and an image of the textile fiber structure 4 recorded by the radiation sensor 8 corresponds substantially to the images recorded according to the embodiments described above.

The device 1 may contain optical elements known to the person skilled in the art, such as lenses, mirrors, apertures, etc., for influencing the radiation 3, 5, 7. For the sake of simplicity, such elements are not shown in FIG. 1.

In one embodiment, the device 1 contains a time-varying optical imaging system which images different parts of the textile fiber structure 4 onto the radiation sensor 8 in chronological order. This can be realized mechanically or electronically. An example of such a time-varying optical imaging system is given in EP-1'961'848 A1 and contains a rotatable polygon mirror for scanning the textile fiber structure 4 line by line. This embodiment requires a time-resolving radiation sensor 8 and a device for assigning the reception time to the corresponding location on the textile fiber structure 4. For this purpose, the radiation sensor 8 does not need to be spatially resolving.

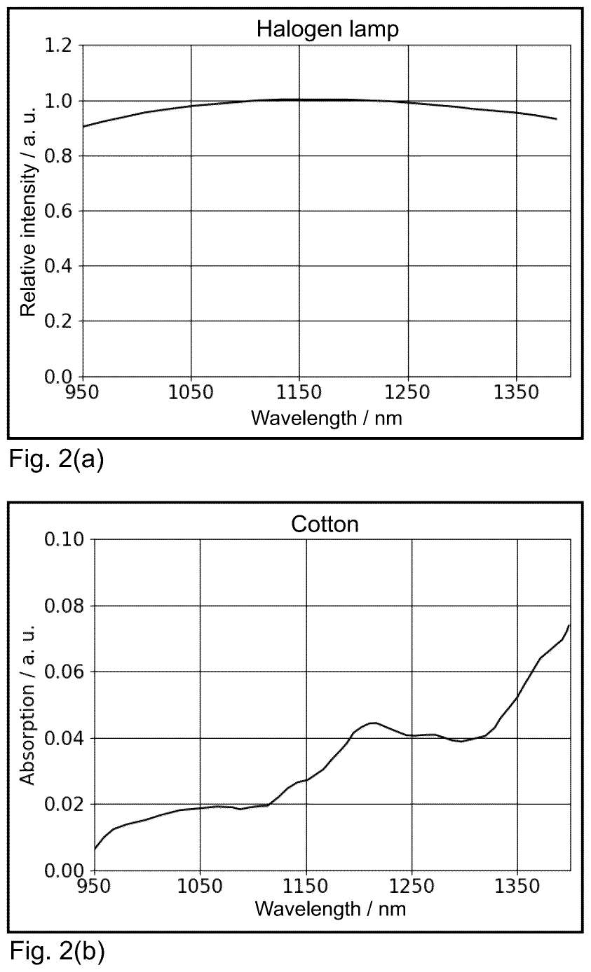

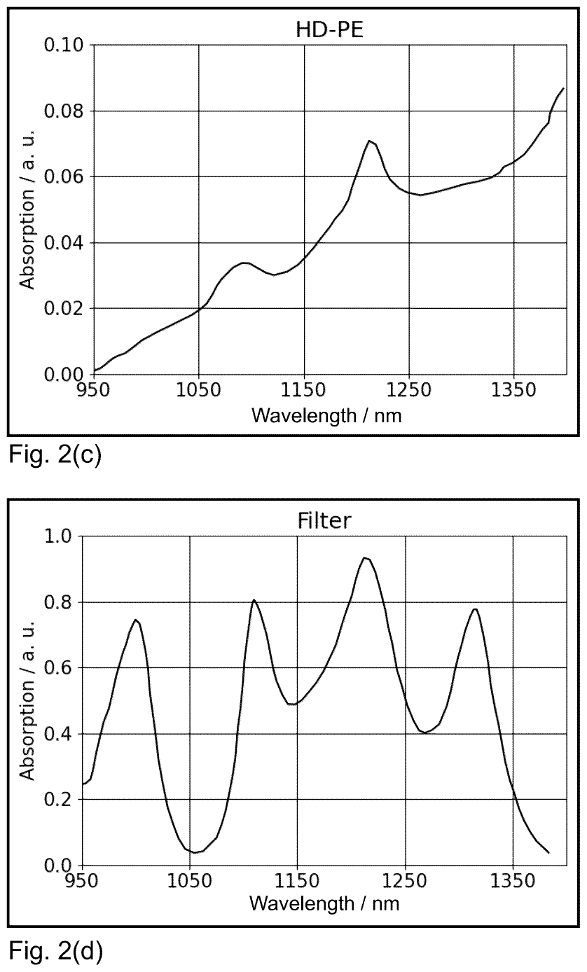

FIG. 2(a) shows an example of the relative intensity of the electromagnetic radiation 3 generated by a halogen lamp 2 as a function of the radiation wavelength λ. In the first spectral band shown (950 nm≤λ≤1400 nm, near and shortwave infrared), the relative intensity decreases monotonously with the radiation wavelength λ. In another spectral band or with other light sources 2, the intensity spectrum can have a different course.

FIG. 2(b) and 2(c) show absorption spectra of cotton, which is a typical textile base material 41, and polyethylene, which can be a foreign material 42, respectively. The respective absorptance is again plotted as a function of the radiation wavelength λ in the same spectral band as in FIG. 2(a).

The spectral properties of the spectral filter 6 are determined by multidimensional variation calculation from the spectral intensity distribution 30 of the radiation source 2 as well as from spectral properties—absorptance, reflectance and/or transmittance—of the base material 41 and the foreign material 42 to be detected. The regression vector resulting from the multidimensional variation calculation contains a weighting for each wavelength in the spectral band under consideration. The weightings correspond to the transmittance or reflectance of the spectral filter 6 for the wavelengths in question. Thus, the spectral filter 6 is optimized for the detection of a particular foreign material 42 in a particular base material 41 and for the determination of the mixing ratio of the two materials 41, 42 when illuminated by a particular radiation source 2. Such methods for designing a spectral filter are known per se; an example can be found in the article “PLS-regression: a basic tool of chemometrics” by S. Wolda, M. Sjöströma, and L. Eriksson, Chemometrics and Intelligent Laboratory Systems, Volume 58, Issue 2, Oct. 28, 2001, pages 109-130.

FIG. 2(d) shows an exemplary transmittance of a spectral filter 6 as a function of the radiation wavelength λ in the same spectral band as in FIG. 2(a)-2(c). In the example shown, the spectral filter 6 has four local maxima (at wavelengths of about λ≈1000 nm, 1110 nm, 1213 nm, and 1317 nm) and three local minima (at wavelengths of about λ≈1055 nm, 1145 nm, and 1268 nm) in the spectral band under consideration (950 nm≤λ≤1400 nm). The spectral filter 6 enhances the differences in absorption of cotton (FIG. 2(b)) and polyethylene (FIG. 2(c)), which is particularly evident at the wavelengths of about λ≈1100 nm, 1210 nm, and 1320 nm in the respective spectra.

The transmission or reflection of the spectral filter 6 is optimized for the two components 41, 42 of the textile fiber structure 4. As a result, those portions of the electromagnetic radiation 5 incident on the spectral filter 6 that result from the interaction of the radiation 5 with the foreign material 42 can pass unattenuated through the spectral filter 6. Components resulting from the base material 41 are attenuated by the spectral filter 6. The signal on the radiation sensor 8 is thus high for the foreign material 42 and low for the base material 41. If the radiation sensor 8 is designed as an image sensor, the foreign material 42 appears as bright image areas and the base material 41 as dark image areas on the image generated by the radiation sensor 8; cf. FIG. 12(b).

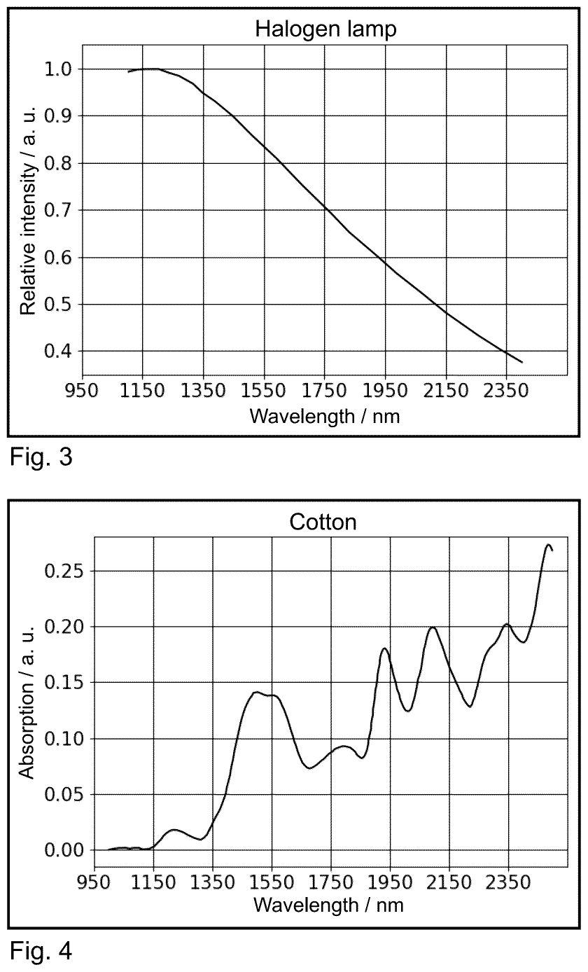

In FIG. 3, the relative intensity of the electromagnetic radiation 3 generated by a halogen lamp 2 is plotted as a function of the radiation wavelength 2 in a second spectral band (approx. 1000 nm≤λ≤2400 nm, near and short-wave infrared), which differs from the first spectral band in FIG. 2.

FIGS. 4-10 show the absorption coefficients of the materials cotton, polyethylene, polypropylene, polyester, polyethylene terephthalate, polyacrylic and viscose (regenerated cellulose) in the second spectral band.

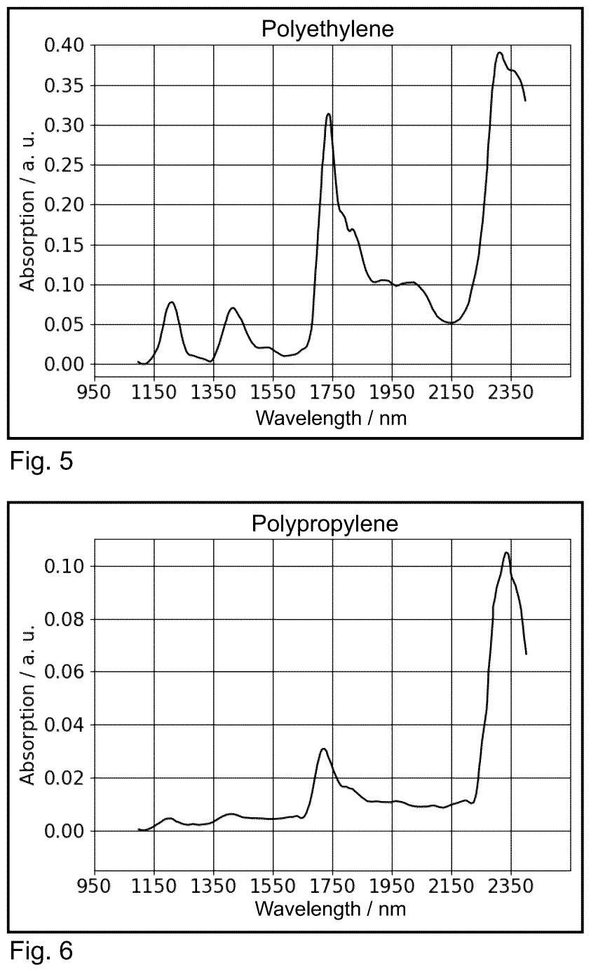

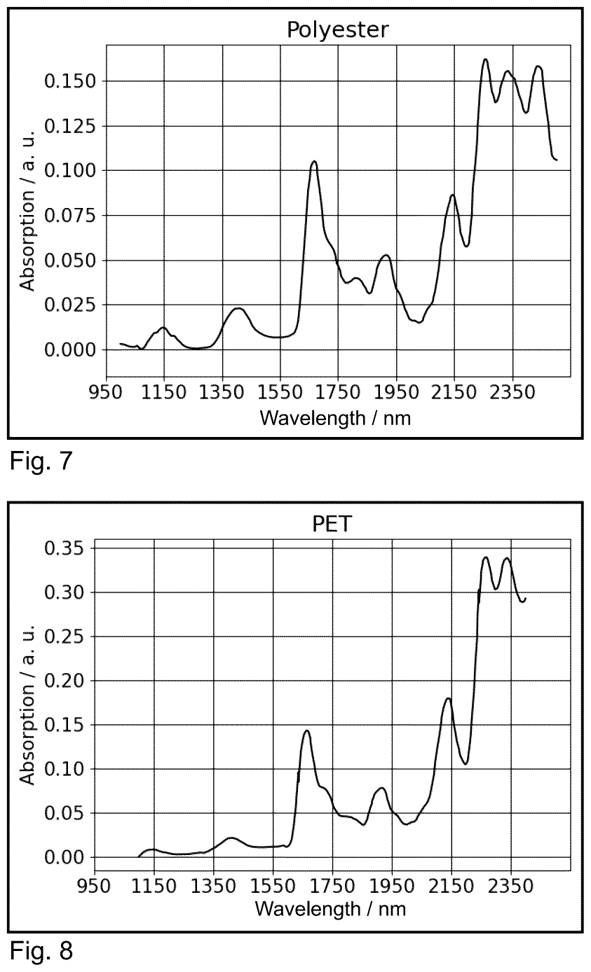

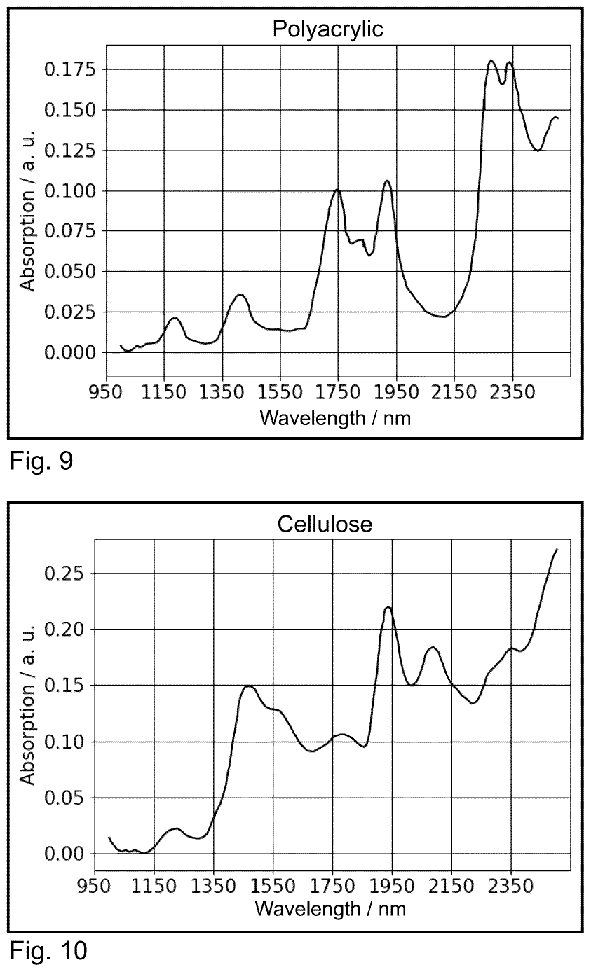

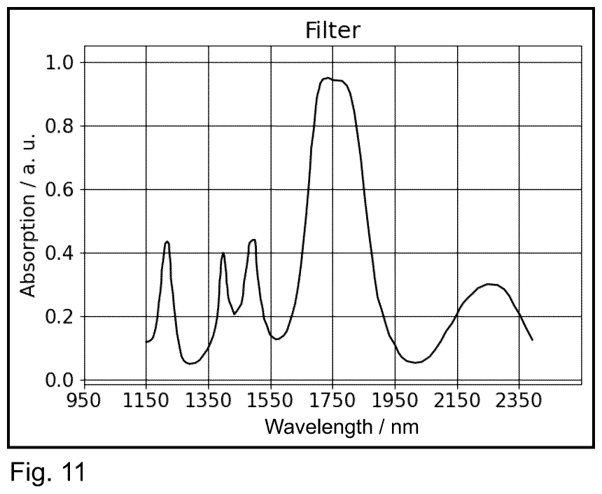

In FIG. 11, an exemplary transmittance of a spectral filter 6 is plotted as a function of the radiation wavelength λ in the second spectral band. This exemplary spectral filter 6 has five local maxima (at wavelengths of approx. λ≈1210 nm, 1400 nm, 1500 nm, 1720 nm and 2260 nm) and four local minima (at wavelengths of approx. λ≈1290 nm, 1440 nm, 1570 nm and 2030 nm) in the second spectral band under consideration (approx. 1000 nm≤λ≤2400 nm). The spectral filter 6 of FIG. 11 is designed for cotton (FIG. 4) as the base material 41 and polyethylene (FIG. 5), polypropylene (FIG. 6), polyester (FIG. 7), polyethylene terephthalate (FIG. 8), polyacrylic (FIG. 9) or polystyrene as the foreign material 42. Such use of one and the same spectral filter 6 for two or more different materials 42 is possible, provided that the spectra of the materials 42 have similar characteristics in the spectral band under consideration. This is the case for the aforementioned foreign materials 42: as FIGS. 5-9 show, the absorption spectra of all these foreign materials 42 have pronounced local maxima in the vicinity of the wavelengths λ≈1700 nm und λ≈2300 nm, in contrast to the base material 41 cotton under consideration (FIG. 4). Accordingly, the spectral filter 6 of FIG. 11 exhibits a pronounced local maximum in the vicinity of λ≈1700 nm and a further local maximum in the vicinity of λ≈2300 nm.

FIG. 12(a) shows a grayscale image of a textile sample with various materials placed on it, recorded with infrared radiation in the spectral band between 1100 nm and 1700 nm. The base material 41 of the sample is a knitted cotton fabric. Pieces of film made of the following foreign materials are placed on it: polystyrene 421, polyethylene 422 and polypropylene 423. The pieces of film 421, 422 and 423 are transparent in the relevant spectral band and are therefore poorly visible in the grayscale image of FIG. 12(a). In practice, they are all the more difficult to recognize in a photographic image in the visible or NIR spectral range. In contrast to FIG. 12(a), in the application example of a fiber clearing device in the blowroom, the base material 41 is not a uniform, flat knitted fabric, but a three-dimensional fiber flock with various shadows, and the foreign material 42 is not a large-area, flat film, but a fiber-like snippet. The invention remedies this.

FIG. 12(b) shows an image of the textile sample of FIG. 12(a) which was taken by a camera 8 of the device 1 according to the invention (see FIG. 1) and subsequently digitally processed. The radiation source 2 used was a halogen lamp with an emission spectrum as shown in FIG. 3. The spectral filter 6 used corresponded to that of FIG. 11. Even in the unprocessed photographic image (not shown), the foreign materials 421, 422, 423 are clearly visible as light areas against a dark background. In order to further emphasize the differences between the base material 41 and the foreign materials 421, 422, 423, the image was subjected to simple digital image processing by defining a brightness threshold below which the pixels were set to “black” and above which the pixels were set to “white”. This produced the binary image in FIG. 12(b), in which the foreign materials 421, 422, 423 stand out clearly from the base material 41.

It is noteworthy that in the example of FIG. 12, the same device 1 according to the invention can be used for three different foreign materials 421, 422, 423. In other words, it is not necessary to design and build a dedicated spectral filter 6 for each foreign material 42; likewise, a single radiation source 2, a single radiation sensor 8 and a single spectral band are sufficient.

It is understood that the present invention is not limited to the embodiments discussed above. With knowledge of the invention, the person skilled in the art will be able to derive further variants which also form part of the subject matter of the present invention.

### LIST OF REFERENCE SIGNS

1 Device according to the invention
2 Radiation source
3 Electromagnetic radiation generated by the radiation source
30 Spectral intensity distribution
4 Textile fiber structure
41 Base material of the textile fiber structure
42 Foreign material in the textile fiber structure
421 Polystyrene
422 Polyethylene
423 Polypropylene
5 Radiation reflected or transmitted by the textile fiber structure
50 Spectral intensity distribution
6 Spectral filter
7 Radiation after the spectral filter
70 Intensity of the detected radiation as a function of the proportion of foreign materials
8 Radiation sensor

1. System, consisting of a textile fiber structure (4) containing two components (41, 42) and a device (1) for investigating the textile fiber structure (4), wherein the device (1) contains,
a radiation source (2) for transmitting electromagnetic radiation (3) in a spectral band in the direction of the textile fiber structure (4) for interaction with the textile fiber structure (4),
a radiation sensor (8) for receiving at least one part of the electromagnetic radiation (7) after interaction with the textile fiber structure (4), and
a spectral filter (6) having spectral properties in the spectral band for filtering at least a part of the electromagnetic radiation (5) before or after interaction with the textile fiber structure (4),
wherein
the transmittance or the reflectance of the spectral filter (6) in the spectral band has at least one local maximum and at least one local minimum, and
the spectral properties of the spectral filter (6) in the spectral band are adapted to the spectral properties of the radiation source (2) and each of the two components (41, 42) such that a radiation intensity received by the radiation sensor (8) is a monotonic function of the mixing ratio of the two components (41, 42).

2. System according to claim 1, wherein the two components (41, 42) of the textile fiber structure (4) are two mutually different elements from the following set: cotton, linen, virgin wool, polyethylene, polypropylene, polyvinyl chloride, polyester; polyacrylic; viscose, modal, lyocell, polyethylene terephthalate, polystyrene, polyamide, acrylonitrile-butadiene-styrene copolymer, polymethyl methacrylate, polyoxymethylene, elastane, aramid, acetate, cupro.

3. System according to claim 2, wherein the two components (41, 42) of the textile fiber structure (4) are a pair from the following set: cotton and polyethylene, cotton and polypropylene, cotton and polyvinyl chloride, cotton and polyester; cotton and polyacrylic; cotton and viscose, cotton and polyethylene terephthalate, cotton and polystyrene, cotton and polyamide, cotton and acrylonitrile-butadiene-styrene copolymer, cotton and polymethyl methacrylate, cotton and polyoxymethylene.

4. Device (1) for investigating a textile fiber structure (4) containing two components (41, 42), comprising:
a radiation source (2) for transmitting electromagnetic radiation (3) in a spectral band in the direction of the textile fiber structure (4) for interaction with the textile fiber structure (4),
a radiation sensor (8) for receiving at least a portion of the electromagnetic radiation (7) after interaction with the textile fiber structure (4), and
a spectral filter (6) having spectral characteristics in the spectral band for filtering at least a portion of the electromagnetic radiation (5) before or after the interaction with the textile fiber structure (4),
wherein
the two components (41, 42) of the textile fiber structure (4) are two mutually different elements from the following set: cotton, linen, virgin wool, polyethylene, polypropylene, polyvinyl chloride, polyester; polyacrylic;
viscose, modal, lyocell, polyethylene terephthalate, polystyrene, polyamide, acrylonitrile-butadiene-styrene copolymer, polymethyl methacrylate, polyoxymethylene, elastane (EL), aramid, acetate, cupro,
the transmittance or reflectance of the spectral filter (6) in the spectral band has at least one local maximum and at least one local minimum, and
the spectral properties of the spectral filter (6) in the spectral band are matched to the spectral properties of the radiation source (2) and each of the two components (41, 24) such that a radiation intensity received by the radiation sensor (8) is a monotonic function of the mixing ratio of the two components (41, 42).

5. Device (1) according to claim 4, wherein the two components (41, 42) of the textile fiber structure (4) are a pair from the following set: cotton and polyethylene, cotton and polypropylene, cotton and polyvinyl chloride, cotton and polyester; cotton and polyacrylic; cotton and viscose, cotton and polyethylene terephthalate, cotton and polystyrene, cotton and polyamide, cotton and acrylonitrile-butadiene-styrene copolymer, cotton and polymethyl methacrylate, cotton and polyoxymethylene.

6. System according to claim 1, wherein the at least one local maximum lies at the wavelength or wavelengths of the electromagnetic radiation (3) at which the absolute value of the difference of the absorptance, the transmittance, or the reflectance of the two components (41, 42) has a local maximum.

7. System according to claim 1, wherein the transmittance or the reflectance of the spectral filter (6) in the spectral band has at least two local maxima and local minima each.

8. System according to claim 1, wherein the spectral filter (6) is designed as a reflection filter or as a transmission filter.

9. System according to claim 1, wherein the spectral filter (6) is designed as an interference filter.

10. System according to claim 1, wherein the spectral filter (6) is integrated into the radiation sensor (8).

11. System according to claim 1, wherein the spectral band is in the wavelength range between 300 nm and 2200 nm, and preferably in the wavelength range between 700 nm and 1900 nm.

12. System according to claim 1, wherein the spectral band has a width between 100 nm and 1500 nm and preferably between 300 nm and 500 nm.

13. System according to claim 1, wherein the spectral properties of the spectral filter (6) in the spectral band are matched to the spectral properties of the radiation source (2) and each of the two components (41, 24) such that a radiation intensity received by the radiation sensor (8) is a linear function of the mixing ratio of the two components (41, 42).

14. System according to claim 1, containing an optical imaging system for imaging the textile fiber image (4) onto the radiation sensor (8), wherein the radiation sensor (8) is spatially resolving and formed either as a digital camera with a two-dimensional image converter or as a one-dimensional line sensor.

15. System according to claim 1, containing a time-varying optical imaging system which images various locations of the textile fiber structure (4) onto the radiation sensor (8) one after the other over time, wherein the radiation sensor (8) is time-resolving.

16. Use of the system of claim 14 for detecting a foreign material (42) in a base material (41), wherein the foreign material and the base material (41) are the two components of the textile fiber structure (4).

17. Use of the system of claim 1 for determining a mixing ratio of the two components (41, 42) of the textile fiber structure (4).

18. Method (1) for investigating a textile fiber structure (4) containing two components (41, 42), the method comprising the steps of:
electromagnetic radiation (3) in a spectral band is transmitted from a radiation source (2) in the direction of the textile fiber structure (4),
at least a part of the electromagnetic radiation (3) interacts with the textile fiber structure (4),
at least a part of the electromagnetic radiation (7) is received by a radiation sensor (8) after interacting with the textile fiber structure (4), and
at least a part of the electromagnetic radiation (5) is filtered by a spectral filter (6) with spectral properties in the spectral band before or after interacting with the textile fiber structure (4),
wherein
the spectral filter (6) is selected such that
its transmittance or its reflectance in the spectral band has at least one local maximum and at least one local minimum, and
its spectral properties in the spectral band are matched to the spectral properties of the radiation source (2) and each of the two components (41, 42) in the textile fiber structure (4) such that a radiation intensity received by the radiation sensor (8) is a monotonic function of the mixing ratio of the two components (41, 42)

19. Method according to claim 18, wherein the spectral band is in the wavelength range between 300 nm and 2200 nm, and preferably in the wavelength range between 700 nm and 1900 nm.

20. Method according to claim 18, wherein the spectral band has a width between 100 nm and 1500 nm and preferably between 200 nm and 500 nm.

21. Method according to claim 18, wherein the two components (41, 42) of the textile fiber structure (4) are two elements different from each other from the following set: cotton, linen, virgin wool, polyethylene, polypropylene, polyvinyl chloride, polyester; polyacrylic; viscose, modal, lyocell, polyethylene terephthalate, polystyrene, polyamide, acrylonitrile-butadiene-styrene copolymer, polymethyl methacrylate, polyoxymethylene, elastane, aramid, acetate, cupro.

22. Method according to claim 21, wherein the two components (41, 42) of the textile fiber structure (4) are a pair from the following set: cotton and polyethylene, cotton and polypropylene, cotton and polyvinyl chloride, cotton and polyester; cotton and polyacrylic; cotton and viscose, cotton and polyethylene terephthalate, cotton and polystyrene, cotton and polyamide, cotton and acrylonitrile-butadiene-styrene copolymer, cotton and polymethyl methacrylate, cotton and polyoxymethylene.

23. Application of the method according to claim 18 for detecting a foreign material (42) in a base material (41), wherein the foreign material (42) and the base material (41) are the two components of the textile fiber structure (4).

24. Application of the method according to claim 18 for determining a mixing ratio of the two components (41, 42) of the textile fiber structure (4).
